(19) United States
(12) Patent Application Publication
(10) Pub. No.: US 2017/0223914 A1
(43) Pub. Date: Aug. 10, 2017

(54) SIMILAR PERFORMANCE FROM SEEDS WITH EPIGENETIC TRAITS

(71) Applicant: Michael E Fromm, Lincoln, NE (US)

(72) Inventor: Michael E Fromm, Lincoln, NE (US)

(21) Appl. No.: 15/419,700

(22) Filed: Jan. 30, 2017

Related U.S. Application Data
(60) Provisional application No. 62/291,084, filed on Feb. 4, 2016.

(51) Int. Cl. A01H 1/02; A01G 7/00

(52) U.S. Cl. CPC A01H 1/02; C12Y 306/03; C12Y 201/01072; A01G 7/00

(57) ABSTRACT

Methods for using seeds comprising an epigenetic trait for reproducibly producing seed lots for sale with similar agronomic performance over multiple years and production cycles are provided.

### CROSS-REFERENCE TO RELATED APPLICATIONS

None

### INCORPORATION OF SEQUENCE LISTING

None

### STATEMENT REGARDING FEDERALLY SPONSORED RESEARCH OR DEVELOPMENT

This invention did not use Government Support.

### BACKGROUND

Epigenetic modifications to improve plant performance in the field is a new emerging area. Epigenetic-based traits can be less stable than traditional genetic changes when propagated for multiple generations, giving rise to stability of performance concerns when using epigenetic-based traits in commercial crop production. This epigenetic instability could be a barrier to commercializing plants with reproducible improved agronomic performance when the trait or traits are dependent in part on epigenetic modifications.

Plant genomes contain relatively large amounts of 5-methylcytosine (5 meC; Kumar et al. 2013 J Genet 92(3): 629-666). Other than silencing transposable elements and repeated sequences, the biological roles of 5 meC and its transgenerational stability are still emerging. Intercrossing a low methylation mutant plant with a normally methylated plant resulted in heritable changes in DNA methylation in the plant genome that affected some plant phenotypic traits (Cortijo et al. 2014 Science. 2014 Mar. 7; 343(6175): 1145-8). Over expression of Arabidopsis MET 1, a DNA methyltransferase predominantly responsible for CG maintenance methylation, in Arabidopsis resulted in plants that flower earlier (U.S. Pat. Nos. 6,011,200 and 6,444,469). Gene mutations affecting DNA methylation include those described (Stroud et al., Cell 2013 152(1-2)352-64) that can alter DNA methylation patterns in progeny.

### SUMMARY

The present invention provides a method of reproducibly obtaining multiple crop production cycles with similar agronomic performance over multiple years from a parent plant or line comprising an epigenetically unstable trait, wherein continued annual production of seeds leads to loss or alteration of the epigenetic trait(s) due to transgenerational epigenetic instability as the number of generations increases. The present invention also provides a method of reproducibly obtaining multiple crop production cycles with similar agronomic performance over multiple years from one or more parent plants or plant lines with an epigenetic trait whose stability for multiple generations of plant life cycles is not known. The invention also provides reproducible crop performance in the final seed lots to be sold after multiple generations of growing plants and harvesting seeds to increase the number of seeds for sale across different production cycles in different years.

In a non-limiting embodiment, sufficient seeds to initiate annual production cycles to produce seeds lots for sale over a period of at least two years to at least ten years are produced from one or more parent plants comprising an epigenetic-based trait, mixed to form a founder seed batch, and frozen for use in different annual production cycles initiated over multiple years.

Each aliquot of the seed batch contains sufficient numbers of founder seeds to provide a starting population of plants for a production cycle occurring over several generations of seed amplification such that similar average trait performance is achieved in the seeds for sale each production cycle. A larger number of seeds in the starting aliquots provides a better statistical representation of the founder seed batch and thereby provides a more consistent agronomic performance in the final seed lots. Mixtures of seeds from different founder lines could be mixed without departing from the scope of method. Seeds from founder lines stored by frozen and non-frozen methods of storage could be mixed to comprise a seed mixture with at least 50% of the seeds from frozen storage without departing from the scope of method. The present invention provides a method of providing epigenetically similar starting aliquots of epigenetically modified seeds for obtaining consistent agronomic performance over multiple production cycles.

Most crop seeds can be stored at about −18 C to −20 C or lower temperatures and retain high levels of germination for at least 10 years. Adjusting the seed moisture levels prior to freezing improves seed viability and germination. Seeds are often initially air dried in air with 30% or less humidity. Desiccants such as silica gel are used to dry the seeds to a moisture content of 15% or less, preferably 5% to 10%, or most preferably a range of 5% to 7% moisture content. Ultradrying to a moisture content of 1% to 4% can also be used.

A relatively small number of founder seeds can be frozen as the starter populations of each production cycle if several generations of seed amplification are performed subsequently from each aliquot of frozen seeds for each production cycle. Each production cycle results in seeds for sale in a specific year. In a non-limiting embodiment, the number of seed amplification generations amplified by self-pollination is at least 2 and less than 8. Preferably the number of generations amplified by self-pollination is 2, 3, 4, or 5 generations. A limited number of generations limits the loss of performance of an epigenetic trait over multiple generations.

A sufficient number of seeds in each aliquot allows each aliquot of starter seeds to produce a population of progeny seeds that have similar average trait and/or yield behavior over each production cycle. A number of founder seeds sufficient to produce the final number of seeds for a desired number of plants acres, given the amplification factor provided in the desired number of generations, is a useful aliquot size. For example, if a number of seeds sufficient to plant 1 M acres of plants is desired, a calculation involving the final number of seeds, the number of generations, and the seed increase each generation, will determine the initial minimum number of founder seeds needed from an aliquot of frozen seeds. More than the minimum number of seeds can be used. In a non-limiting embodiment, the number of seeds in an aliquot is at least 10, at least 25, at least 50, preferably at least 100, and most preferably at least 150 seeds. There is no upper limit on the number of seeds in an aliquot except that larger aliquots require larger amounts of freezer space. The size of the seed batch and the seed aliquot size is chosen by the individual using the method, the number of generations in the production cycle, the amplification in each generation, and the final number of seeds desired.

A non-limiting example of an epigenetically-modified inbred plant line is an inbred plant line derived from a Msh1-suppressed ancestral plant or plants and methods of producing them. See U.S. patent application Ser. No. 13/462,216, filed May 2, 2012, U.S. patent application Ser. No. 14/454,518, filed Aug. 7, 2014, U.S. Provisional Patent Application Ser. No. 61/882,140, filed Sep. 25, 2013, and U.S. Provisional Patent Application Ser. No. 62/275,602, filed Jan. 6, 2016, which are each incorporated herein by reference in their entireties. Some, but not all, plant lines derived from Msh1 suppressed plants or from grafting normal shoots or scions to rootstocks suppressed for Msh1 or derived from Msh1 suppressed plants, contain epigenetic modifications that do not persist for an indefinite number of generations. For such epigenetically modified plant lines a method to reproducibly produce seed lots that can be used to produce crops over multiple years with similar agronomic performance is desirable and useful.

A non-limiting example of the present invention is for the production of ‘hybrid-like’ seeds for sale. In the production of hybrid-like seeds, an aliquot of frozen seeds from an epigenetically unstable plant line are grown (first parental line) and crossed to a second parental plant line. This second parental plant line can be genetically and/or epigenetically the same or different than the first parent line. After this initial cross of the two parent plants, the F1 progeny are self-pollinated for several generations, preferably 3 to 5 generations, and the seeds from the last generation of plants are harvested, treated, stored, and sold according to standard commercial seed production methods.

Yet another non-limiting embodiment of the present invention is for the production of seeds from grafted plants. In the production of seeds initiated by a grafting step, an aliquot of frozen seeds from an epigenetically modified line or variety are grown to be used as a rootstock in a graft to a second parental plant line or variety used as the shoot or scion in a graft. This second parental plant line or variety can be epigenetically and/or genetically the same or different than rootstock line or variety. After this initial grafting of the two parent plants, the S1 progeny are harvested and self-pollinated for several generations, preferably 2 to 5 generations, and the seeds from the last generation of plants are harvested, treated, stored, and sold according to standard commercial seed production methods. Seeds after the grafting step of the aforementioned example can be frozen as a post-grafting founder seeds as an alternative method of obtaining consistent agronomic performance from epigenetic traits.

### BRIEF DESCRIPTION OF THE DRAWINGS

FIG. 1 provides a list of the steps for producing, drying, and freezing a seed batch of epigenetically modified seeds and producing seeds for sale with similar performance over multiple years from aliquots of a frozen seed batch. Optional procedures are also indicated at the appropriate steps.

### DESCRIPTION

As used herein, the phrase “epigenetically unstable trait” refers to a trait dependent in part on epigenetic modifications including DNA methylation, wherein the epigenetically unstable trait changes its phenotype in at least a subset of progeny plants when a genetically homogeneous (inbred) plant line is propagated by self-pollination for 8 generations or less. For example, a yield increase that is dependent in part upon DNA methylation modifications wherein a yield increase present initially in a plant line is diminished in some or most of the plants of said plant line after 8 generations or less of self-pollination.

As used herein, the phrase “epigenetic trait” refers to a trait dependent in part on epigenetic modifications. For example, a yield increase that is dependent in part upon DNA methylation modifications.

As used herein, the phrases “seed batch” or “founder seed batch” refers to the total group of seeds to be stored for a set of production cycles. A seed batch can be subdivided into aliquots, one aliquot for each production cycle. Alternatively the desired number of seeds can be obtained from the seed batch without prior subdivision into subsets. A seed batch can be prepared from different stages of a production cycle as long as the number of seeds in the seed batch are sufficient for the number of production cycles required when starting at that step in the production cycle. A seed batch can be from a single inbred line or more than one inbred line or from one or more plant lines before reaching an inbred status.

As used herein, the phrase “founder seeds” refers to the seeds frozen to form a seed batch or founder seed batch as well as aliquots of seeds from a seed batch or founder seed batch.

As used herein, the phrase “seed lot” refers to the seeds produced for sale for a specific year for a plant lineage or lineages (a lineage has a common ancestor or ancestors). Sometimes unsold seeds from a seed lot are stored for sale for one or more years. A seed lot can be derived from a single line or from one or more plant lines prior or after a crossing or grafting step.

As used herein, the phrases “production cycle” (singular) or “production cycles” (plural) refer to producing a seed lot for annual seed sales or producing seed lots for multiple annual seed sales, typically one seed lot for 1 to 2 years of sales. A production cycle typically requires growing several generations of plants over one or more years and all the multiple generations of plants grown for seed amplification to produce a seed lot for sale in a particular year are part of that year's production cycle. The number of generations in a production cycle is the number of generations starting from the first generation grown from the common founder seed batch to the final seed lot for sale. The seeds within the founder seed batch share a common ancestry or common ancestries when seeds with different ancestors are mixed. Seeds from a seed batch can be frozen at different generational steps in different production cycles without departing from the scope of the method. A production cycle can comprise: a) only self-pollinated plants; or b) F1 seeds followed by self-pollination of subsequent generations; or c) seeds from self-pollinated plants and end with F1 seeds when hybrid seeds are to be sold; or seeds for rootstocks in a grafting production cycle; or seeds produced from grafted parental plants.

As used herein, the phrase “molecular markers diagnostic for desired epigenetic modifications” refers to molecular markers that directly or indirectly measure DNA methylation or epigenetic modifications at specific chromosomal regions in a sequence specific manner. These methods include those that directly measure DNA methylation levels, indirect measurements such as sRNA levels, and indirect measurements of gene expression such as mRNA levels (or the resulting cDNA) of genes whose expression has been affected by epigenetic modifications.

As used herein, the term “aliquot” (singular) or “aliquots” (plural) refers to a subset or subsets, respectively, of a seed batch of sufficient size for starting or continuing the production cycle from that point forward.

As used herein, the phrase “plant line” refers to multiple plants within a plant generation and/or multiple generations of plants derived from a common ancestor or two common ancestors.

As used herein, the phrase “inbred” refers to a plant line sufficiently homozygous to have progeny with similar phenotypes. Typically an inbred plant has been self-pollinated for at least three generations or has been generated from a double haploid method to have a high degree of genetic and/or epigenetic homozygosity.

As used herein, the term “inbred” refers to a plant line that is predominantly homozygous genetically and epigenetically due to at least several generations of self-pollination and selection for relatively uniform progeny phenotypes or is derived from a double haploid plant.

As used herein, the term “generation” (singular) or “generations” (plural) refers to one or several complete life cycle (an initial seed is grown to a mature plant that produces seeds), wherein each generation of plants produces a larger number of seeds upon harvest.

As used herein, the term “progeny” refers to a first, second, third, or later generations of plants derived from a parent or ancestor plant.

As used herein, the term “F1” refers to seeds produced from a cross of two parents or plants grown from said seeds.

As used herein, the term “F2” refers to seeds produced from self-pollination of plants grown from F1 seeds or plants grown from F2 seeds.

As used herein, the term “F3” refers to seeds produced from self-pollination of plants grown from F2 seeds or plants grown from F3 seeds.

As used herein, the term “Fn” refers to seeds produced from self-pollination of plants grown from F(n−1) seeds or plants grown from Fn seeds.

### Epigenetic Markers

DNA, mRNA or its derived cDNA, or sRNA can serve as useful markers that provide information about the underlying epigenetic status for an epigenetic-based trait such as increased yield or stress tolerance.

In general, the process for obtaining DNA methylation measurements from a seed or plant of interest is as follows. Obtain plants cells or tissue(s) from a plant seed or plant, wherein the cells or tissues are from the embryo or tissues or pollen derived from the embryo, i.e., not maternal or endosperm tissues. Exemplary tissues include but are not limited to scutellum, leaves, immature tassels, immature ears, stems, roots, and pollen. Suitable methods for obtaining plant cells or tissues include, but are not limited to, drilling, slicing, cutting, laser-removal, automated methods, and the like, to obtain one or more cells from the target seed or tissue. (U.S. Pat. Nos. 7,611,842; 7,703,238; 7,941,969; 9,027,278; 9,003,696). The cell or cells are disrupted to release the nuclear DNA. Disruption methods include, but are not limited to, grinding, extrusion, freeze-thaw, alkali treatment, squeezing, or the like, with either fresh, frozen, dried or hydrated cells or tissues. The disrupted cell or cells are suspended or extracted into buffer solution suitable for DNA extraction. The solution containing DNA from the plant cell or cells can be analyzed directly or preferably further processed to further purify the plant DNA.

The isolated plant DNA is treated to deaminate non-methylated C nucleotides within one or more DNA strands. An exemplary treatment is bisulfite treatment. Bisulfite treated DNA is then analyzed to determine whether a specific DNA sequence position contains a C or T base, wherein the original DNA sequence prior to bisulfite treatment was a C or methylated-C nucleotide. The proportion of C to T bases at a specific DNA sequence position measures the degree of DNA methylation at that specific position. Exemplary methods include DNA sequencing or single base detection methods such as fluorescent nucleotide extension assays, sequence-specific ligation assays, Taqman assays and similar assays known to those skilled in the art of measuring DNA methylation and/or single nucleotide polymorphism differences. The measured amount of methylation at one or more specific DNA positions is used for the computational analysis or analyses described herein.

In certain embodiments, DNA methylation of chromosomal regions can be identified by identifying small RNAs that are up or down regulated in the test plants (in comparison to reference plants). This method is based in part on identification of small interfering RNAs that direct or maintain DNA methylation of specific gene targets by RNA-directed DNA methylation (RdDM). The RNA-directed DNA methylation (RdDM) process has been described (Chinnusamy V et al. Sci China Ser C-Life Sci. (2009) 52(4): 331-343). Any applicable technology platform can be used to compare small RNAs in the test and reference plants, including, but not limited to, microarray-based methods (Franco-Zorilla et al. Plant J. 2009 59(5):840-50), deep sequencing based methods (Wang et al. The Plant Cell 21:1053-1069 (2009)), and the like. Any applicable technology platform can be used to compare small RNAs in the test and reference plants, including, but not limited to: microarray-based methods (Franco-Zorilla et al. Plant J. 200959(5):840-50); deep sequencing based methods (Wang et al. The Plant Cell 21:1053-1069(2009); Wei et al., Proc Natl Acad Sci USA. 2014 Feb. 19, 111(10): 3877-3882; Zhai et al., Methods. 2013 Jun. 28. pii: S1046-2023(13)00237-5. doi: 10.1016/j.ymeth.2013.06.025 or J. Zhai et al., Methods (2013), http://dx.doi.org/10.1016/j.ymeth.2013.06.025); U.S. Pat. No. 7,550,583; U.S. Pat. No. 8,399,221; U.S. Pat. No. 8,399,222; U.S. Pat. No. 8,404,439; U.S. Pat. No. 8,637,276; Rosas-Cárdenas et al., (2011) Plant Methods 2011, 7:4; Moyano et al., BMC Genomics. 2013 Oct. 11; 14:701; Eldem et al., PLoS One. 2012; 7(12):e50298; Barber et al., Proc Natl Acad Sci USA. 2012 Jun. 26; 109(26):10444-9; Gommans et al., Methods Mol Biol. 2012; 786:167-78; and the like.

Any applicable technology platform can be used to compare the DNA methylation status of chromosomal loci in test and reference plants. Applicable technologies for identifying chromosomal loci with changes in their methylation status include, but not limited to, methods based on immunoprecipitation of DNA with antibodies that recognize 5-methylcytidine, methods based on use of methylation dependent restriction endonucleases and PCR such as McrBC-PCR methods (Rabinowicz, et al. Genome Res. 13: 2658-2664 2003; Li et al., Plant Cell 20:259-276, 2008), sequencing ofbisulfite-converted DNA (Frommer et al. Proc. Natl. Acad. Sci. U.S.A. 89 (5): 1827-31; Tost et al. BioTechniques 35 (1): 152-156, 2003), methylation-specific PCR analysis of bisulfite treated DNA (Herman et al. Proc. Natl. Acad. Sci. U.S.A. 93 (18): 9821-6, 1996), deep sequencing based methods (Wang et al. The Plant Cell 21:1053-1069 (2009)), methylation sensitive single nucleotide primer extension (MsSnuPE; Gonzalgo and Jones Nucleic Acids Res. 25 (12): 2529-2531, 1997), fluorescence correlation spectroscopy (Umezu et al. Anal Biochem. 415(2):145-50, 2011), single molecule real time sequencing methods (Flusberg et al. Nature Methods 7, 461-465), high resolution melting analysis (Wojdacz and Dobrovic (2007) Nucleic Acids Res. 35 (6): e41), and the like.

Additional applicable technologies for identifying chromosomal loci with changes in their DNA methylation status include, but not limited to, the preparation, amplification and analysis of Methylome libraries as described in U.S. Pat. No. 8,440,404; using Methylation-specific binding proteins as described in U.S. Pat. No. 8,394,585; determining the average DNA methylation density of a locus of interest within a population of DNA fragments as described in U.S. Pat. No. 8,361,719; by methylation-sensitive single nucleotide primer extension (Ms-SNuPE), for determination of strand-specific methylation status at cytosine residues as described in U.S. Pat. No. 7,037,650; a method for detecting a methylated CpG-containing nucleic acid present in a specimen by contacting the specimen with an agent that modifies unmethylated cytosine and amplifying the CpG-containing nucleic acid using CpG-specific oligonucleotide primers as described in U.S. Pat. No. 6,265,171; an improved method for the bisulfite conversion of DNA for subsequent analysis of DNA methylation as described in U.S. Pat. No. 8,586,302; for treating genomic DNA samples with sodium bisulfite to create methylation-dependent sequence differences, followed by detection with fluorescence-based quantitative PCR techniques as described in U.S. Pat. No. 8,323,890; a method for retaining methylation pattern in globally amplified DNA as described in U.S. Pat. No. 7,820,385; a method for detecting cytosine methylations DNA as described in U.S. Pat. No. 8,241,855; a method for quantification of methylated DNA as described in U.S. Pat. No. 7,972,784; a highly sensitive method for the detection of cytosine methylation patterns as described in U.S. Pat. No. 7,229,759; additional methods for detecting DNA methylation changes are described in U.S. Pat. No. 7,943,308 and U.S. Pat. No. 8,273,528.

Examples of suitable plants for the present invention, provided that a specific plant species has seeds that can be dried and frozen, include those from the genera Medicago, Lycopersicon, Brassica, Cucumis, Solanum, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, and Nicotiana; the family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum; corn (Zea mays), canola (Brassica napus, Brassica rapa ssp.), Brassica species useful as sources of seed oil, alfalfa (Medicago sativa), rice (Oryza sativa), rye (Secale cereale), sorghum (Sorghum bicolor, Sorghum vulgare), millet (for example, pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia spp.), almond (Prunus amygdalus), sugar beets (Beta vulgaris), sugarcane (Saccharum spp.), oats, barley, vegetables, ornamentals, and conifers; vegetables plants, for example, tomatoes (Lycopersicon esculentum), lettuce (for example, Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo); ornamental plants, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum; leguminous plants, for example, guar, locust bean, fenugreek, soybean, garden beans, cowpea, mungbean, lima bean, fava bean, lentils, chickpea, peanuts (Arachis sp.), crown vetch (Vicia sp.), hairy vetch, adzuki bean, lupine (Lupinus sp.), trifolium, common bean (Phaseolus sp.), field bean (Pisum sp.), clover (Melilotus sp.) Lotus, trefoil, lens, and false indigo; forage and turf grass, for example, alfalfa (Medicagos sp.), orchard grass, tall fescue, perennial ryegrass, creeping bent grass, and redtop.

Examples of crop plants include, but are not limited to, alfalfa, corn, soybeans, beans, wheat, rice, cotton, canola/rapeseed, tomatoes, peanuts, peppers, barley, oats, rye, sorghum, millet, and tobacco.

Examples of plant traits or characteristics include improved yield, delayed flowering, non-flowering, increased biotic stress resistance, increased abiotic stress resistance, enhanced lodging resistance, enhanced growth rate, enhanced biomass, enhanced tillering, enhanced branching, delayed flowering time, and delayed senescence; agronomic traits (flowering time, days to flower, days to flower-post rainy, days to flowering; fungal disease resistance; grain related traits: (Grain dry weight, grain number, grain number per square meter, Grain weight over panicle. seed color, seed luster, seed size); growth and development stage related traits (basal tillers number, days to harvest, days to maturity, nodal tillering, plant height, plant height); infloresence anatomy and morphology trait (threshability); Insect damage resistance; leaf related traits (leaf color, leaf midrib color, leaf vein color, flag leaf weight, leaf weight, rest of leaves weight); mineral and ion content related traits (shoot potassium content, shoot sodium content); panicle, pod, or ear related traits (number of panicles and seeds, harvest index, panicle weight); phytochemical compound content (plant pigmentation); xii) spikelet anatomy and morphology traits (glume color, glume covering); stem related trait (stem over leaf weight, stem weight); and miscellaneous traits (stover related traits, metabolised energy, nitrogen digestibility, organic matter digestibility, stover dry weight); various seed quality traits including improvements in either the compositions or amounts of oil, protein, or starch in the seed; increased biomass, non-flowering, male sterility, digestability, seed filling period, maturity (either earlier or later as desired), reduced lodging, and plant height (either increased or decreased as desired) improved resistance to biotic plant stress; stress imposed by plant fungal pathogens, plant bacterial pathogens, plant viral pathogens, insects, nematodes, and herbivores; resistance to fungal pathogens including, an Alternaria sp., an Ascochyla sp., a Boirylis sp., a Cercospora sp., a Colletotrichum sp., a Diaporthe sp., a Diplodia sp., an Erysiphe sp., a Fusarium sp., Gaeumanomyces sp., Helminthosporium sp., Macrophomina sp., a Nectria sp., a Peronospora sp., a Phakopsora sp., Phialophora sp., a Phoma sp., a Phymatotrichum sp., a Phytophthora sp., a Plasmopara sp., a Puccinia sp., a Podosphaera sp., a Pyrenophora sp., a Pyricularia sp, a Pythium sp., a Rhizoctonia sp., a Scerotium sp., a Sclerotinia sp., a Septoria sp., a Thielaviopsis sp., an Uncinula sp, a Venturia sp., and a Verticillium sp.; bacterial pathogens including Erwinia sp., a Pseudomonas sp., and a Xanthamonas sp., resistance to insects including aphids and other piercing/sucking insects such as Lygus sp., lepidoteran insects such as Armigera sp., Helicoverpa sp., Heliothis sp., and Pseudoplusia sp., and coleopteran insects such as Diabroticus sp., resistance to nematodes including Meloidogyne sp., Heterodera sp., Belonolaimus sp., Ditylenchus sp., Globodera sp., Naccobbus sp., and Xiphinema sp.

Other traits include yield improvements are improvements in the yield of a plant line relative to one or more parental line(s) under non-stress conditions. Non-stress conditions comprise conditions where water, temperature, nutrients, minerals, and light fall within typical ranges for cultivation of the plant species. Such typical ranges for cultivation comprise amounts or values of water, temperature, nutrients, minerals, and/or light that are neither insufficient nor excessive. In certain embodiments, such yield improvements are improvements in the yield of a plant line relative to parental line(s) under abiotic stress conditions. Such abiotic stress conditions include, but are not limited to, conditions where water, temperature, nutrients, minerals, and/or light that are either insufficient or excessive. Abiotic stress conditions would thus include, but are not limited to, drought stress, osmotic stress, nitrogen stress, phosphorous stress, mineral stress, heat stress, cold stress, and/or light stress. In this context, mineral stress includes, but is not limited to, stress due to insufficient or excessive potassium, calcium, magnesium, iron, manganese, copper, zinc, boron, aluminum, or silicon. In this context, mineral stress includes, but is not limited to, stress due to excessive amounts of heavy metals including, but not limited to, cadmium, copper, nickel, zinc, lead, and chromium.

### EXAMPLES

The following examples are included to demonstrate various embodiments. However, those of skill in the art should, in light of the present disclosure, appreciate that many changes can be made in the specific embodiments which are disclosed and still obtain a like or similar result without departing from the spirit and scope of the invention.

### Example 1. Drying Seeds Before Freezing

Adjusting seed moisture levels prior to freezing improves seed viability and germination. Seeds are initially air dried in air with 30% or less humidity. Desiccants such as silica gel are then used to dry the seeds to a moisture content of about 10%, preferably about 8%, or most preferably a range of about 5% to 7% moisture content prior to freezing. Ultradrying to a 1% to 4% moisture can also be used. Seeds are kept in an air tight container to minimize absorbing moisture from the external air. Seed containers are preferably of glass with rubber gaskets or plastic-lined metal containers with plastic or rubber gaskets.

### Example 2. Freezing Seeds

Temperatures of −10 C or lower can be used to freeze seeds for multi-year storage. An optimal temperature range is about −18 C to −20 C. Lower temperatures can also be used. Seeds with low moisture content (see Example 1) in air-resistant containers such as glass or metal (See Example 1) are stored at about −18 C to −20 C for long term storage of the seeds (long term is one to fifteen years). Preferably the freezer should have minimal temperature fluctuations, maintaining the target temperature within a degree to several degrees.

### Example 3. Production of Seeds Starting from Frozen Seeds

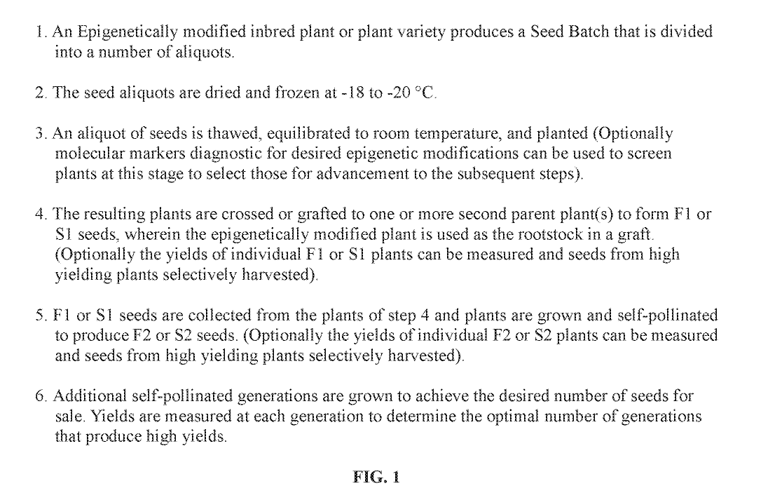

FIG. 1 provides a non-limiting example of a production cycle to produce large amounts of seeds for sale. In FIG. 1, the epigenetically unstable parent inbred line produces sufficient seeds to produce a seed batch (Step 1). The seed batch contains a number of aliquots corresponding to at least the number of production cycles desired. The seeds in a seed batch are air dried, then dried with silica gel desiccant to a moisture of 5% to 7%, and frozen at −20 C in a sealed glass container (FIG. 1 Step 2). The number of seeds in each aliquot is set by the producer. In the present example about 150 seeds are present in each aliquot.

The beginning of a representative production cycle is when an aliquot of seeds are thawed and are planted in soil (FIG. 1 Step 3). The plants from these seeds will be used for the initial crossing or grafting step. Optionally individual plants can be analyzed with molecular markers that directly or indirectly measure epigenetic modifications that are associated with the epigenetic-dependent trait such as higher yields and/or higher stress tolerance relative to plants lacking this epigenetic-dependent trait. Plants with preferred markers present are selected for further steps.

The second parent for crossing or grafting is also planted. For crossing either parent can be the pollen source, while for grafting, the epigenetically modified plant is used as a rootstock in a graft (FIG. 1 Step 4). The second parent can be genetically identical or similar to the first parent or the parents can be genetically and epigenetically different, according to the goal of the person performing the production cycle. Harvesting seeds from the crossed parents will provide enough F1 (crossed) or S1 (grafted and self-pollinated) or F1 (grafted and crossed) seeds for planting the desired amount of field space (FIG. 1 Step 5; The scale of each step can be adjusted to fit the number of seeds desired or needed at each generation).

The F1 or S1 seeds are grown to produce F2 or S2 seeds (FIG. 1 Step 6). Optionally the yields of the individual F2 or S2 plants can be measured as described in U.S. Provisional Patent Application Ser. No. 62/275,602, filed Jan. 6, 2016, which is incorporated herein by reference in its entirety, and seeds from the higher yielding F2 or S2 plants selected if desired. The harvested F2 or S2 plants produce F3 or S3 seeds.

F3 or S3 seeds are grown to produce F4 seeds that are harvested (FIG. 1 Step 7) (Optionally the yields of the F3 or S3 plant families can be measured as described in U.S. Provisional Patent Application Ser. No. 62/275,602, filed Jan. 6, 2016, which is incorporated herein by reference in its entirety, and seeds from the higher yielding F3 families selected if desired). Additional generations can be grown to produce the desired amount of seeds for sale (FIG. 1 Step 6). The exact amplification of seeds obtained in each generation will vary as can the number of acres planted at each generation, according to the amount of seeds available and the number of acres desired at each step. The seed amplification amounts obtained in each generation can vary without departing from the scope and purposes described in this example.

Seeds can be frozen at any of the stages. Larger amounts of seeds require large amounts of freezer storage. Freezing seeds at the stages more advanced than the inbred seeds has the advantage of shortening the time required for the subsequent amplification steps required before seed sales and the producer can decide which generation to freeze.

### Example 4. Varieties Propagated by Only Self-Pollination

The steps of Example 3 are followed except that plants are never crossed: every generation is fertilized by self-pollination. A plant line produced by self-pollination only can be considered a variety.

### Example 5. Production of a Hybrid Crop

The steps of Example 3 are followed except that plants of every generation is fertilized by self-pollination except the last generation producing the seed lot for sale. This last generation is crossed in a manner appropriate for that crop species. For example methods of producing hybrid corn, hybrid tomatoes, hybrid canola, hybrid rice, hybrid sorghum, hybrid alfalfa, and other hybrid crops known to those skilled in the art.

What is claimed is:

1. A method of producing seeds comprising an epigenetically unstable trait to have similar agronomic performance over at least two production cycles comprising:
a) producing a batch of seeds comprising an epigenetically unstable trait and of a sufficient number of seeds for completing at least two production cycles; and,
b) growing plants from said seed batch of step (a) for at least a first and second production cycle each comprising at least 1 plant generation and less than 8 plant generations, wherein at least 50% of the seeds for producing at least one plant generation have been stored at −10° C. or less.

2. The method of claim 1, wherein the epigenetically unstable trait is unstable over two or more plant generations.

3. The method of claim 1, wherein the temperature of step (b) is about −18° C. to −20° C.

4. The method of claim 1, wherein the amount of seeds in a seed batch is at least 50, 100, 500, or 1,000 seeds.

5. The method of claim 1, wherein the amount of seeds produce at the conclusion of the production cycle is sufficient to plant 10,000, 100,000, or 1,000,000 acres.

6. The method of claim 1, wherein the number of plant generations for a production cycle of step (b) is 2 to 3, 4 to 5, or 6 to 7 generations.

7. The method of claim 1, wherein the seeds are derived from an ancestor plant suppressed for Msh1 function or from a grafted plant comprising a rootstock suppressed for Msh1 function or derived from an ancestor plant suppressed for Msh1 function.

8. The method of claim 1, wherein the seeds of said seed batch of step (a) are derived from an ancestor plant suppressed for expression or over-expressing of a gene affecting DNA methylation levels.

9. The method of claim 1, wherein the seeds of said seed batch of step (a) are derived from an ancestor plant expressing a transgenic DNA methylase.

10. The method of claim 1, wherein the seeds of said seed batch of step (a) are derived from an ancestor plant suppressed for expression of a gene encoding a plastid localized protein and which affects DNA methylation levels.

11. The method of claim 1, wherein said the seeds of step (b) stored at −10° C. or less are from the first, second, or third plant generations of said second production cycle of step (b).

12. The method of claim 1, wherein the last generation of plants of the production cycle is crossed to produce hybrid seeds for sale.

13. The method of claim 1, wherein the all the generations of plants of the production cycle are self-pollinated at each plant generation.

14. The method of claim 1, wherein the plants grown in step (b) from the seed batch of step (a) are individually analyzed for one or more epigenetic markers; and a subset of the plants are selected for seed production for subsequent plant generations on the basis of the epigenetic marker results.

15. The method of claim 1, wherein the seed batch of step (a) comprises seeds from at least two plants lines mixed either before or after freezing.
